(19) United States
(12) Patent Application Publication
(10) Pub. No.: US 2008/0160100 A1
(43) Pub. Date: Jul. 3, 2008

(54) Method For Stimulating Hair Growth And Regeneration

(76) Inventors: Igor Anatolievich Pomytkin, Moscow (RU); Sergey Pavlovich Soloviev, Moscow (RU)

Correspondence Address: NOTARO AND MICHALOS, 100 DUTCH HILL ROAD, SUITE 110, ORANGEBURG, NY, 10962-2100, US

(21) Appl. No.: 11/817,935

(22) PCT Filed: Mar. 11, 2005
(86) PCT No.: PCT/RU2005/000107

§ 371 Date: Nov. 15, 2007

(51) Int. Cl. A61K 33/00

(52) U.S. Cl. 424/600

(57) ABSTRACT

The invention relates to method for stimulating hair growth and regeneration in a mammal in need thereof, which comprises a step of applying to the skin of said mammal an effective amount of water comprising from about 99.760 to about 99.999% of light isotopologue ¹H₂¹⁶0 and up to 100% of residual isotopologues ¹H₂¹⁷0, ¹H₂¹⁸0, ¹H₂H¹⁶0 ¹H₂H¹⁷0, ¹H₂H¹⁸0, ²H₂¹⁶0, ²H₂¹⁷0, and ²H₂¹⁸0. Preferably, the mammal is a human.

### TECHNICAL FIELD

The invention relates to healthcare. More specifically, the invention relates to the use of water enriched by light water isotopologue ¹H₂¹⁶O for stimulating hair growth and regeneration.

### BACKGROUND ART

In humans, the growth and regeneration of hair are determined principally by the activity of the hair follicles. They function cyclically, and this hair follicle cycle includes three main phases: anagen, catagen, and telogen. The anagen is growth stage of the hair follicle cycle (from 2 to 7 years), the catagen is a stage of the hair cycle characterized by regression and involution of the follicle (2 to 3 weeks) and the telogen is resting stage of the hair cycle (3 to 4 months). At the end of the resting phase, the hairs fall out and another cycle begins. Of the 100,000 to 150,000 scalp hairs on a human adult (regardless of sex), about 90% are in the growing, or anagen, phase, the remaining about 10% are in the telogen phase.

The cyclic regeneration of hairs is under control of numerous factors. It is known that positive regulators of hair growth and regeneration are keratinocyte growth factor (KGF), insulin-like growth factors (IGFs), and hepatocyte growth factor (HGF), while pro-inflammatory cytokines such as TNF-alpha and IL-1beta inhibits hair growth. Stenn K. S. and Paus R. Physiol. Rev. 2001, 81 (1): 449-494.

Hair is often subjected to a wide variety of insults that can cause hair damage and hair loss. These include shampooing, rinsing, drying, heating, combing, styling, perming, colouring, exposure to the chemical substances, radiation therapy, chemotherapy, and etc. Also, hair is often subjected to disorders which include male pattern baldness, alopecia greata, alopecia induced by cancer chemotherapy, hair thinning associated with aging, and etc.

Thus, there is a great need for a safe, effective agent for hairs care that improves hair regeneration and growth with little or no complications and side effects.

U.S. Pat. No. 4,139,619, discloses a topical composition comprising Minoxidil and related iminopyrimidines which stimulates the conversion of vellus hair to terminal hair and increases the rate of growth of terminal hair.

Natural water is a composition of nine water isotopologues (¹H₂¹⁶O, ¹H₂¹⁷O, ¹H₂¹⁸O, ¹H²H¹⁶O, ¹H²H¹⁷O, ¹H²H¹⁸O, ²H₂¹⁶O, ²H₂¹⁷O, and ²H₂¹⁸O) formed by stable isotopes of hydrogen (¹H and ²H) and oxygen (¹⁶O, ¹⁷O, ¹⁸O), wherein the level of light water isotopologue ¹H₂¹⁶O is about 99.7317% (Vienna Standard Mean Ocean Water, VSMOW), and wherein total level of all eight heavy isotopologues comprising at least one heavy isotopes ²H, ¹⁷O, and ¹⁸O is about 0.2683% (e.g. 0.199983% ¹H₂H₂¹⁸O, 0.0372% ¹H₂¹⁷O, 0.031069% ¹H²H¹⁶O, 0.0000623% ¹H²H¹⁸O, and 0.0000116% ¹H²H ¹⁷O). Rothman et al., J. Quant. Spectrosc. Radiat. Transfer, 1998, 60, 665. Rothman et al., J. Quant. Spectrosc. Radiat. Transfer, 2003, 82, p. 9. The abundance of water isotopologues in natural water slightly varies on Earth district and climatic conditions and is expressed typically as the deviation, 6, relative to the international VSMOW standard. The natural water enriched maximally by major light water isotopologue ¹H₂¹⁶O was founded in Antarctica (Standard Light Antarctic Precipitation, SLAP), wherein said δ-values of residual heavy isotopes are δ²H-415.5‰, δ¹⁷O-28.1‰, and δ¹⁸O-53.9‰ that corresponds to the 99.757% level of light water isotopologue ¹H₂¹⁶O. R. van Trigt, Laser Spectrometry for Stable Isotope Analysis of Water Biomedical and Paleoclimatological Applications, 2002, Groningen: University Library Groningen, p. 50. Thus, water with the abundance of light water isotopologue 1H₂¹⁶O more than 99.757% is not found in nature.

Complete depletion of natural water of deuterium-comprising isotopologues (¹H²H¹⁶O, ¹H²H¹⁷O, ¹H²H¹⁸O, ²H₂¹⁶O, ²H₂, and ²H₂¹⁸O) provides water enriched by light water isotopologue ¹H₂¹⁶O to the level never more than 99.76%, since total level of these deuterium-comprising isotopologues in water is below 0.031%. Thus, water with content of light water isotopologue ¹H₂¹⁶O more than 99.76% can be prepared in industrial scale by methods providing depletion of natural water of heavy isotopologues ¹H₂¹⁷O, ¹H₂¹⁸O, ¹H²H¹⁶O, ¹H²H¹⁷O, ¹H²H¹⁸O, ²H₂¹⁶O, ²H₂¹⁷O, and ²H₂¹⁸O.

We discovered that water enriched by light isotopologue ¹H₂¹⁶O to levels more than 99.76% is useful for stimulating hair growth and regeneration in mammals in need thereof.

It is an object of the present invention to provide a method for stimulating hair growth and regeneration in a mammal in need thereof, which comprises a step of applying to the skin of said mammal an effective amount of water comprising from about 99.760 to about 99.999% of light isotopologue ¹H₂¹⁶O and up to 100% of residual isotopologues ¹H₂¹⁷O, ¹H₂¹⁸O, ¹H²H¹⁶O, ¹H²H¹⁷O, ¹H²H¹⁸O, ²H₂¹⁶O, ²H₂¹⁷O, ²H₂¹⁸O.

### BRIEF DESCRIPTION OF THE DRAWINGS

FIG. 1 is a schematic side view of an apparatus for the manufacturing the water comprising from about 99.760 to about 99.999% of light isotopologue ¹H₂¹⁶O and up to 100% of residual isotopologues ¹H₂¹⁷O, ¹H₂¹⁸O, ¹H²H¹⁶O, ¹H²H¹⁷O, ¹H²H¹⁸O, ²H₂¹⁶O, ²H₂¹⁷O, ²H₂¹⁸O.

FIG. 2 is a photo of hematoxylin-and-eosin-stained paraffin sections of skin obtained from mice treated with heavy isotope depleted water (A) or usual water (B) at day 8 after depilation. Scale bars=100 μm.

### DISCLOSURE OF INVENTION

The present invention provides a method for stimulating hair growth and regeneration in a mammal in need thereof, which comprises a step of applying to the skin of said mammal an effective amount of water comprising from about 99.760 to about 99.999% of light isotopologue ¹H₂¹⁶O and up to 100% of residual isotopologues ¹H₂¹⁷O, ¹H₂¹⁸O, ¹H²H¹⁶O, ¹H²H¹⁷O, ¹H²H¹⁸O, ²H₂¹⁶O, ²H₂¹⁷O, ²H₂¹⁸O.

As used herein, the term “isotopologue” is in accordance with IUPAC Compendium of Chemical Terminology 2nd Edition (1997) and refers to a molecular entity that differs only in isotopic composition (number of isotopic substitutions), e.g. ¹H₂¹⁶O, ¹H²H¹⁶O, ¹H₂¹⁸O.

The water of the invention comprising from about 99.760 to about 99.999% of light isotopologue ¹H₂¹⁶O and up to 100% of residual isotopologues ¹H₂⁷⁰O, ¹H₂¹⁸O, ¹H²H¹⁶O, ¹H²H¹⁷O, ¹H²H¹⁸O, ²H₂¹⁶O, ²H₂¹⁷O, ²H₂¹⁸O can be prepared by industrial procedures providing a step of depletion of natural water of heavy isotopologues comprising heavy isotopes ²H, ¹⁷O, and ¹⁸O. In the method of the invention, relative amounts of particular heavy isotopologues could vary depending upon the procedure of the preparing the water of the invention, but the sum of residual heavy isotopologues formed by heavy isotopes should not exceed 0.001 to 0.240%. Accordingly, in the method of the invention, the residual amounts of heavy isotopes in the water of the invention could vary from 0.01 ppm to 155 ppm for ²H, 1 to 360 ppm for ¹⁷O, and 1 to 2000 ppm for ¹⁸O, but the sum of heavy isotopologues formed by these residual heavy isotopes should not exceed 0.001 to 0.240%.

Preferably, the effective amount of the water comprising from about 99.760 to about 99.999% of light isotopologue ¹H₂¹⁶O and up to 100% of residual isotopologues ¹H₂¹⁷O, ¹H₂¹⁸O, ¹H²H¹⁶O, ¹H²H¹⁷O, ¹H²H¹⁸O, ²H₂¹⁶O, ²H₂¹⁷O, ²H₂¹⁸O is 1 to 3000 g per mammalian subject.

Preferably, the effective amount of water comprising from about 99.760 to about 99.999% of light isotopologue ¹H₂¹⁶O and up to 100% of residual isotopologues ¹H₂¹⁷O, ¹H₂¹⁸O, ¹H²H¹⁶O, ¹H²H¹⁷O, ¹H²H¹⁸O, ²H₂¹⁶O, H₂¹⁷O, ²H₂⁸O is applied for 1 day or longer.

Nonexclusive examples of mammals of the invention include humans and animals such as dogs and cats. Preferably, the mammal is a human.

In practicing the method of the invention, the effective amount of water comprising from about 99.760 to about 99.999% of light isotopologue ¹H₂¹⁶O and up to 100% of residual isotopologues ¹H₂¹⁷O, ¹H₂¹⁸O, ¹H²H¹⁶O, ¹H²H¹⁷O, ¹H²H¹⁸O, ²H₂¹⁶O, ²H₂¹⁷O, ²H₂¹⁸O can be applied onto a mammal skin in a variety of topical dosage form. Such forms include, but are not limited to, gels, sprays, syrups, drops, lotions, creams, and solutions. For example, topical dosage form (e.g. gel) is applied on skin of the mammal in need thereof, and is preferably left on the skin for a period of at least about 15 minutes, more preferably at least about 30 minutes, even more preferably at least about 1 hour, most preferably for at least several hours, e.g., up to about 12 hours. This method can be reapplied from 1 to about 5, preferably from 1 to 3 times per day.

The preferred effect of treatment for hair using the method of the invention is damaged hair regeneration and hair growth stimulation.

Herein and after, the term “light water” refers to water comprising from about 99.760% to about 99.999% of light isotopologue ¹H₂¹⁶O and up to 100% of residual isotopologues ¹H₂¹⁷O, ¹H₂¹⁸O, ¹H²H¹⁶O, ¹H²H¹⁷O, ¹H²H¹⁸O, ²H₂¹⁶O, ²H₂¹⁷O, ²H₂¹⁸O.

Light water can be prepared by industrial procedures, for example, by highly-effective distillation of natural water.

The following examples are presented to demonstrate the invention. The examples are illustrative only and are not intended to limit the scope of the invention in any way.

### EXAMPLE 1

This example demonstrates the method for producing light water of the invention.

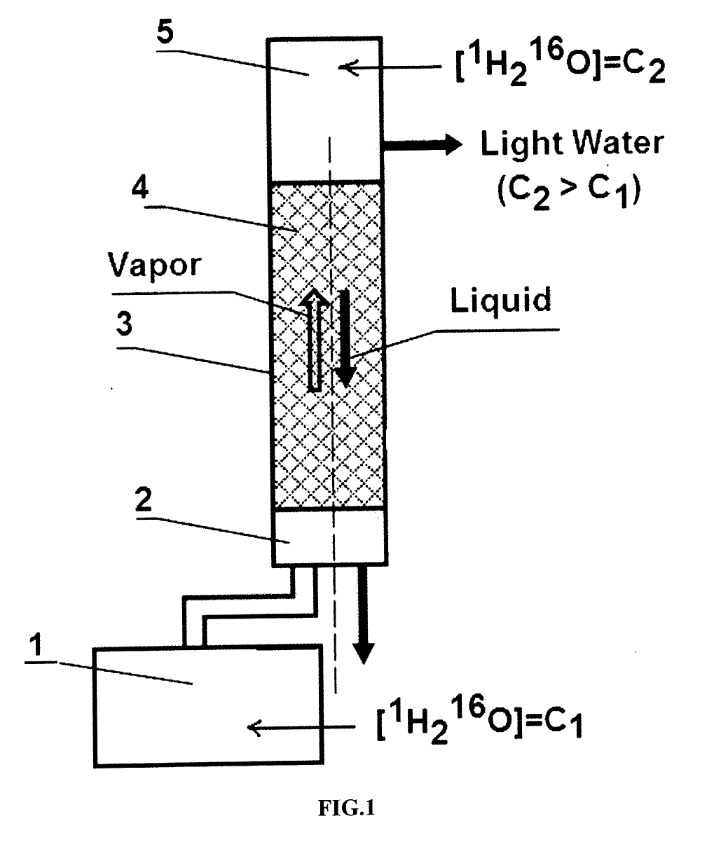

Light water comprising 99.99% of light isotopologue ¹H₂¹⁶O and up to 100% of residual isotopologues (¹H₂¹⁷O, ¹H₂¹⁸O, ¹H²H¹⁶O, ¹H²H¹⁷O, ¹H²H¹⁸O, ²H₂¹⁶O, ²H₂¹⁷O, ²H₂¹⁸O) for use in methods of the invention is prepared by distillation of natural water comprising 99.73% of light isotopologue ¹H₂¹⁶O and up to 100% of residual isotopologues (¹H₂¹⁷O, ¹H₂¹⁸O, ¹H²H¹⁶O, ¹H²H¹⁷O, ¹H²H¹⁸O, ²H₂¹⁶O, ²H₂¹⁷O, ²H₂¹⁸O) with using the apparatus of FIG. 1 under temperature 60° C. and pressure 0.2 bars. The process of the distillation comprises evaporating natural water comprising 99.73% (C₁) of light isotopologue ¹H₂¹⁶O in boiling means 1 to produce water vapor; supplying the water vapor to the bottom 2 of distillation column 3; carrying out vapor-liquid contact between a descending liquid and an ascending vapor mainly on the surface of the contact device 4 (e.g. structured or random packing) within the distillation column, at which time the liquid and the vapor flow in mutually opposite directions over the surface of the contact device along a main flow direction which is along a direction of the column axis; condensing water vapor with concentration of light isotopologue ¹H₂¹⁶O 99.99% (C₂) on condenser 5 installed on upper bound of the distillation column 3; and collecting a part of condensate as condensed raw light water comprising 99.99% of light isotopologue ¹H₂¹⁶O (C₂>C₁). Resulted light water comprising 99.99% of light isotopologue ¹H₂¹⁶O is used in methods of the invention.

### EXAMPLE 2

This example shows the stimulation of hair growth and regeneration.

Materials. Water (99.73% of light isotopologue ¹H₂¹⁶O, control) or light water (99.99% of light isotopologue ¹H₂¹⁶O) was used.

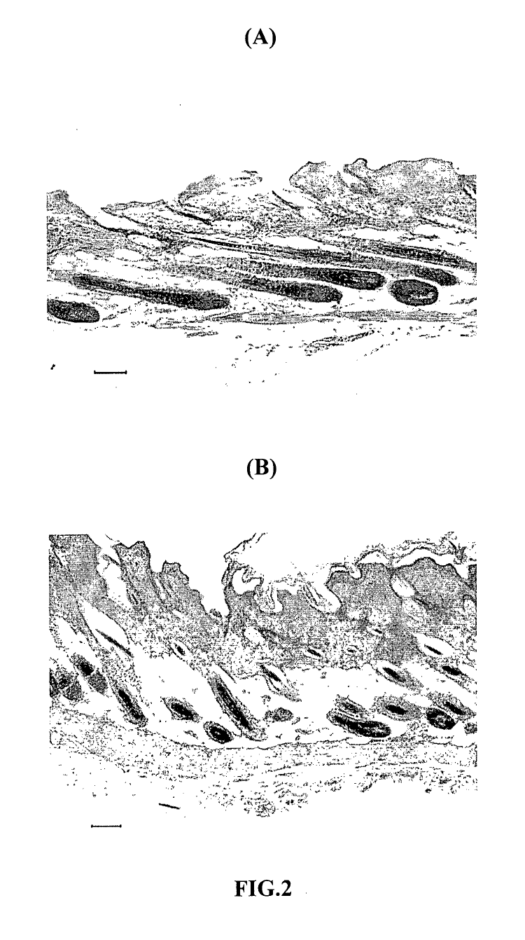

Treatment. Local areas on the back of C57B1 male mice were depilated. Then, control water or light was applied in the amount of 1 g on depilated areas (about 1 cm²) for 1 min once a day during six days. At day 8 after depilation, skin biopsy was taken and hematoxylin-and-eosin-stained paraffin sections were prepared. Histological analysis of the paraffin sections demonstrates increased size of hair bulbs and hair shaft in mice treated with light water as compared to control (FIG. 2). By day 21, it was observable that the treated by light water mice had a hair growth rate greater than that of the mice in the control group. The depilated areas on the back of each mouse was peeled and photographed. The photographic pictures obtained were imaged, using an image processor, whereby the percentage of the hair-grown area relative to the total area of the depilated skin was obtained to be the degree of hair growth. Data are presented in Table 1 as degree of hair growth mean ±S D (n=3) in percent.

Table 1 shows that light water is effective for stimulating hair growth and regeneration as compared to the control.

TABLE 1
Effect of light water on hair growth and regeneration.

| | The degree of hair growth, in |
|---|---|
| Treatment | % of control |
| Control water | 43 ± 8 |
| Light water | 88 ± 8* |

*Differs significantly of control (p < 0.05)

We claim:

1. A method for stimulating hair growth and regeneration in a mammal in need thereof, which comprises a step of applying to the skin of said mammal an effective amount of water comprising from about 99.760 to about 99.999% of light isotopologue ¹H₂¹⁶O and up to 100% of residual isotopologues ¹H₂¹⁷O, ¹H₂¹⁸O, ¹H²H¹⁶O, ¹H²H¹⁷O, ¹H²H¹⁸O, ²H₂¹⁶O, ²H₂¹⁷O, ²H¹⁸O.

2. The method according to claim 1, wherein the effective amount water comprising from about 99.760 to about 99.999% of light isotopologue ¹H₂¹⁶O and up to 100% of residual isotopologues ¹H₂¹⁷O, ¹H₂¹⁸O, ¹H²H¹⁶O, ¹H²H¹⁷O, ¹H²H¹⁸O, ²H₂¹⁶O, ²H₂¹⁸O, H₂H₂¹⁸O is 1 to 3000 g per mammalian subject.

3. The method according to claim 1, wherein said mammal is a human.

4. The method according to claim 1, wherein said water is applied for 1 day or longer.
